(19) United States
(12) Patent Application Publication
(10) Pub. No.: US 2019/0374248 A1
(43) Pub. Date: Dec. 12, 2019

(54) TROCAR CANNULA WITH A GUIDANCE VALVE

(71) Applicant: Alcon Inc., Fribourg (CH)

(72) Inventors: Reto Grueebler, Schaffhausen (CH); Thomas Linsi, Shaffhausen (CH); Philipp Schaller, Stein am Rhein (CH)

(21) Appl. No.: 16/419,349

(22) Filed: May 22, 2019

Related U.S. Application Data
(60) Provisional application No. 62/681,341, filed on Jun. 6, 2018.

(51) Int. Cl. A61B 17/34; A61F 9/007; A61B 17/00

(52) U.S. Cl. CPC A61B 17/3421; A61B 2090/0811; A61B 17/00234; A61F 9/00736

(57) ABSTRACT

Various embodiments are generally directed to a trocar cannula for providing a surgical instrument with access to the interior of an eye, such as a soft tip cannula used in ocular surgery, for instance. Some embodiments are particularly directed to a trocar cannula that facilitates alignment of a surgical instrument with an axis of the cannula with a guidance valve. In one or more embodiments, the trocar cannula may keep one or more portions of the surgical instrument straight as the surgical instrument utilizes the trocar cannula to access the interior of an eye. Many embodiments are directed to a trocar cannula that reduces the amount of intraocular pressure loss when a surgical instrument utilizes the trocar cannula to access the interior of an eye.

### PRIORITY CLAIM

This application claims the benefit of priority of U.S. Provisional Patent Application Ser. No. 62/681,341 titled “Trocar Cannula With A Guidance Valve,” filed on Jun. 6, 2018, whose inventors are Reto Grueebler, Thomas Linsi and Philipp Schaller, which is hereby incorporated by reference in its entirety as though fully and completely set forth herein.

### BACKGROUND

Generally, surgical instruments are tools or devices designed to perform specific actions involved in carrying out desired effects during surgery or operations. Sometimes a trocar cannula may be used to provide a surgical instrument with access to a surgical site, such as the interior of an eye. Typically, surgical instruments are used in ophthalmic surgery. Ophthalmic surgery typically includes performing an operation on an eye or its adnexa. Often ophthalmic surgeries utilize a probe. Further, these surgeries may include operations on the anterior portions of the eye as well as operations on the posterior portions of the eye. In various embodiments, ophthalmic surgery may be performed on a patient for therapeutic purposes.

### BRIEF DESCRIPTION OF THE DRAWINGS

FIGS. 1A and 1B illustrate an exemplary trocar cannula according to one or more embodiments described herein.

FIG. 2 illustrates an exemplary operating environment of a trocar cannula according to one or more embodiments described herein.

FIGS. 3A-3D illustrate an exemplary process of inserting a cannula through a trocar cannula according to one or more embodiments described herein.

FIG. 4 illustrates an exemplary trocar cannula according to one or more embodiments described herein.

FIG. 5 illustrates an exemplary trocar cannula according to one or more embodiments described herein.

FIG. 6 illustrates an exemplary trocar cannula in conjunction with a cannula according to one or more embodiments described herein.

FIG. 7 illustrates an exemplary trocar cannula according to one or more embodiments described herein.

FIG. 8 illustrates an exemplary flow diagram of a method according to one or more embodiments described herein.

### DETAILED DESCRIPTION

Various embodiments are generally directed to a trocar cannula for providing a surgical instrument with access to the interior of an eye, such as a soft tip cannula used in ocular surgery, for instance. Some embodiments are particularly directed to a trocar cannula that facilitates alignment of a surgical instrument with an axis of the cannula with a guidance valve. Many embodiments are directed to a trocar cannula that reduces the amount of intraocular pressure loss when a surgical instrument utilizes the trocar cannula to access the interior of an eye. In one or more embodiments, for example, an apparatus for ocular surgery may include a trocar cannula with a penetrating portion and a receiving portion coupled via a hub. In various embodiments, the penetrating portion may be inserted into an eye to provide a surgical instrument with access to the interior of an eye via the receiving portion. In some embodiments, a guidance valve may be included in the receiving portion to guide alignment of the surgical instrument with an axis of the trocar cannula. In one or more embodiments, the guidance valve may limit equalization between an intraocular pressure of the eye and an ambient pressure external to the eye when the surgical instrument utilizes the trocar cannula to access the interior of the eye.

Some challenges facing trocar cannulas include difficult and time-consuming procedures to insert a surgical instrument into a trocar cannula without inhibiting and/or damaging the surgical instrument. The challenges may result from an inability to guide proper alignment of a surgical instrument with a trocar cannula. For instance, the surgical instrument may include a soft or flexible tip that can become kinked when inserted into a trocar cannula with improper alignment. In some such instances, if the surgical instrument continues to be inserted into the trocar cannula without removing the kink, then surgical complications may occur, including damage to the trocar cannula and/or damage to the surgical instrument. In various embodiments, this may cause a surgeon to go through multiple attempts before successfully inserting a surgical instrument into a trocar cannula.

Adding further complexity, when a trocar cannula is inserted into an eye and/or when a surgical instrument utilizes the trocar cannula to access the eye, undue intraocular pressure may be lost or poorly maintained. Additionally, this issue may be compounded when multiple attempts to insert a surgical instrument into the trocar cannula are required. In various embodiments, loss of intraocular pressure may lead to surgical complications, such as a detached retina. These and other factors may result in unreliable trocar cannulas with limited flexibility, deficient performance, and safety concerns. Such limitations can reduce the capabilities, usability, and applicability of the trocar cannula, contributing to inefficient devices with limited abilities.

Various embodiments described herein include a trocar cannula with a guidance valve that is shaped to promote alignment of a surgical instrument with an entry axis of a trocar cannula. For instance, the guidance valve may include a funnel shape to guide alignment of a surgical instrument with the entry axis of the trocar cannula to access a surgical site. In some instances, the guidance valve may include a membrane with a concave surface shaped to guide axial alignment of a surgical instrument with the trocar cannula. In many embodiments described herein, the guidance valve may limit equalization between a pressure at the surgical site and a pressure external to the surgical site, such as equalization between an intraocular pressure of an eye and an ambient pressure external to the eye. For instance, the guidance valve may include an opening that deforms to limit equalization between the intraocular pressure and the ambient pressure when the trocar cannula accesses the interior of the eye and/or the surgical instrument utilizes the trocar cannula to access the interior of the eye. In some such instances, the opening may comprise one or more slits in a membrane. In some embodiments, the guidance valve may include a membrane with a convex surface exposed to the intraocular pressure and the intraocular pressure pushes the convex surface against the surgical instrument. In various embodiments, the intraocular pressure may push the convex surface and the surgical instrument against each other. In various such embodiments, this may result in a self-sealing valve (e.g., via pressure). In these and other ways one or more of the trocar cannulas described herein may function in a safe and efficient manner to achieve better performing trocar cannulas, resulting in several technical effects and advantages.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well-known structures and devices are shown in block diagram form in order to facilitate a description thereof. The intention is to cover all modification, equivalents, and alternatives within the scope of the claims.

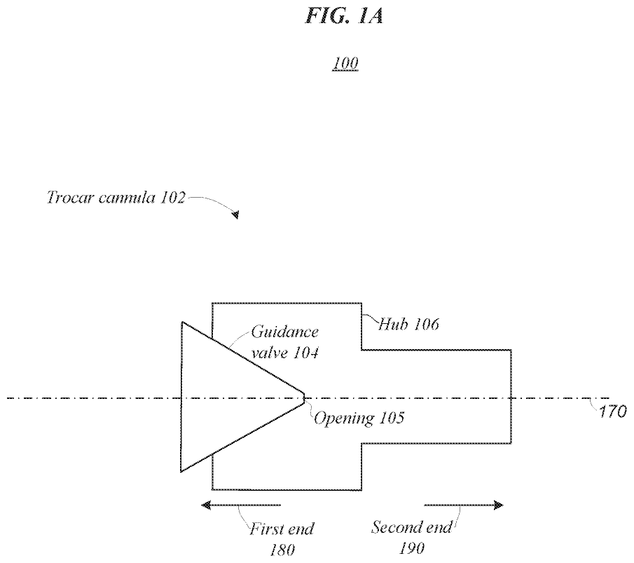

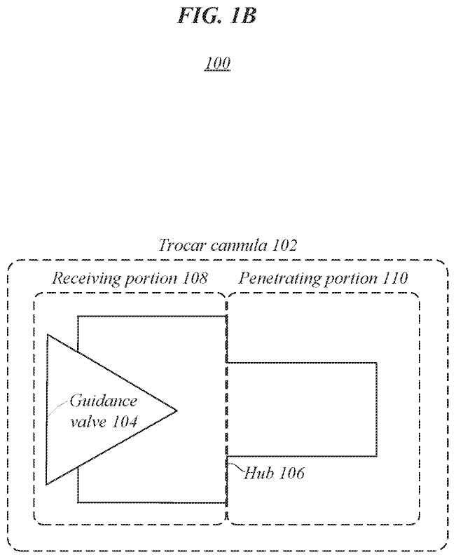

FIGS. 1A and 1B illustrate an embodiment of an operating environment 100 that may be representative of various embodiments. Operating environment 100 may include a trocar cannula 102. In one or more embodiments, trocar cannula 102 may include a valved trocar cannula. In various embodiments, trocar cannula 102 may include a guidance valve 104 and a hub 106. As shown in FIG. 1B, hub 106 may connect a receiving portion 108 with guidance valve 104 and a penetrating portion 110 of trocar cannula 102. In some embodiments, the penetrating portion 110 may be used to gain and/or provide access to a surgical site, such as the interior of an eye. For example, the penetrating portions 110 may be inserted into an eye and enable surgical instruments, such as a soft tip cannula or a soft tip backflush, to pass through the trocar cannula along entry axis 170 and access the interior of the eye. In some embodiments, the penetrating portion 110 may have an end (e.g., a tapered or pointed end) configured to pierce the eye. In some embodiments (e.g., as seen in FIGS. 1A-5 and 7), the penetrating portion 110 may have a non-tapered end. In these embodiments, the trocar cannulas 102 with a non-tapered penetrating portion 110 may be fitted onto a trocar (e.g., with an end that extends past the penetrating portion 110 and is configured to pierce the eye). The trocar may pierce the eye for placement of the trocar cannula 102 and then withdrawn (leaving the trocar cannula in place in the eye). In one or more embodiments described herein, guidance valve 104 may be shaped to guide a surgical instrument inserted in the receiving portion 108, proximate first end 180, such that it aligns with entry axis 170 prior to exiting opening 105, passing hub 106, and exiting second end 190. Embodiments are not limited in this context.

In various embodiments, trocar cannula 102 may include a tubular structure of different radii connected by hub 106. For instance, receiving portion 108 may include a tubular section of a first outer diameter and penetrating portion 110 may include a tubular section of a second outer diameter that is smaller than the first outer diameter. In one or more embodiments, guidance valve 104 may align a surgical instrument with entry axis 170 such that a surgical instrument with a soft or flexible tip can be passed through trocar cannula 102 without becoming kinked, tangled and/or blocked. For example, in prior trocar cannulas, a soft tip cannula or backflush may become kinked or blocked at an opening of a valve or where a hub couples the first diameter to the second diameter. In many embodiments, guidance valve 104 may keep at least one portion of the surgical instrument straight. For instance, guidance valve 104 may keep a soft tip of a trocar cannula straight. In some such instances, this may prevent the soft tip from kinking.

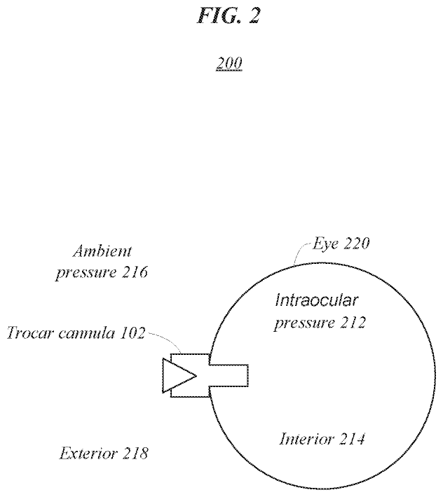

In some embodiments described herein, guidance valve 104 may prevent or limit equalization of different pressures to which the first and second ends 180,190 are exposed. For instance, the first end 180 may be exposed to an ambient pressure and the second end 190 may be exposed to an intraocular pressure (see e.g., FIG. 2). In one or more embodiments, guidance valve 104 may prevent or reduce leakage of a higher pressure at the surgical site to a lower pressure external to the surgical site, such as loss of intraocular pressure within an eye. In one or more embodiments described herein, the first end 180 may be referred to as the proximal end and the second end 190 may be referred to as the distal end. In various embodiments, opening 105 may include one or more slits or holes. In some embodiments, opening 105 may self-seal when a surgical instrument is not inserted therethrough. For instance, opening 105 may self-seal in response to exposure to an intraocular pressure. In many embodiments, guidance valve 104 may include a membrane with opening 105 that conforms to surgical instruments inserted therethrough to prevent or limit equalization of different pressures to which the first and second ends 180, 190 are exposed.

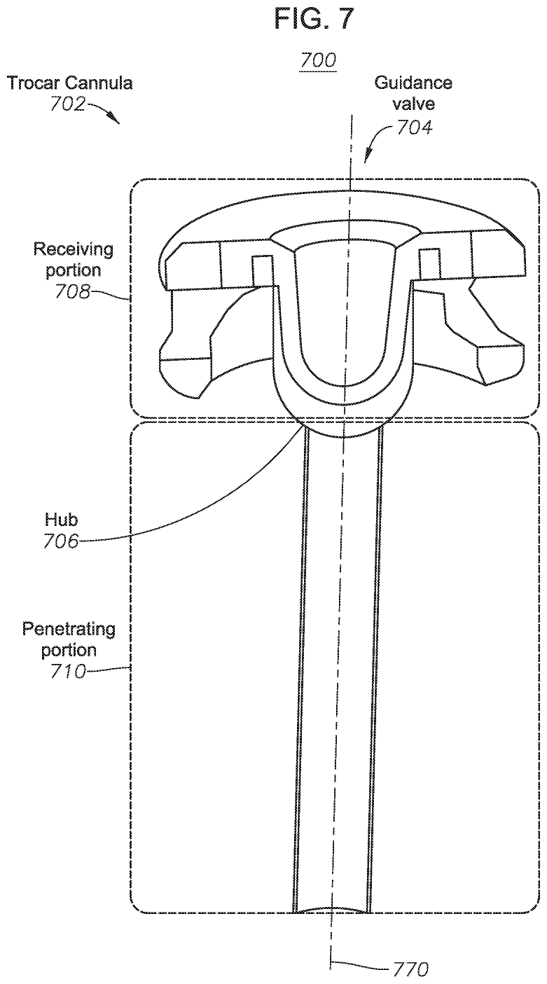

In some embodiments, the membrane may include a spherical shape. In some of such embodiments, the spherical shape may utilize intraocular pressure to limit an amount of intraocular pressure lost. In various embodiments, backpressure on the guidance valve 104 created by the intraocular pressure may support self-sealing of the guidance valve with a surgical instrument, such as a backflush or soft tip cannula (see e.g., FIG. 7). Additionally, backpressure on the guidance valve 104 created by intraocular pressure may support self-sealing of the guidance valve with itself when no surgical instrument is inserted therethrough. In some embodiments, guidance valve 104 may utilize an elastic material to conform with surgical instruments passed therethrough.

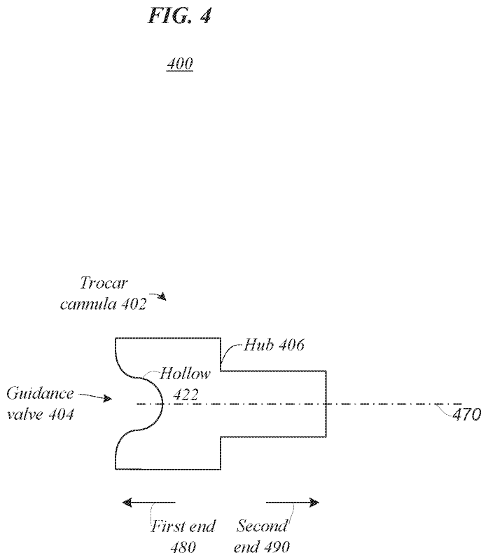

In one or more embodiments, insertion of a surgical tool, such as a soft tip cannula, through trocar cannula 102 may be simplified and expedited with guidance valve 104. In some embodiments, guidance valve 104 may include a membrane with a cylindrical hollow aligned with entry axis 170 (see e.g., FIG. 4). In some such embodiments, a diameter of the cylindrical hollow may decrease when moving towards opening 105. When a surgical instrument is inserted in the hollow proximate first end 180, the hollow provides guidance such that the surgical instrument can pass through opening 105 and past hub 106 without becoming kinked or damaged. In one or more embodiments, opening 105 may include or be referred to as a valve membrane. For example, opening 105 may include one or more slits in a membrane comprising at least a portion of guidance valve 104. In some embodiments, the membrane may include one or more of silicon, polyurethane, and polyimide. Other materials are also contemplated.

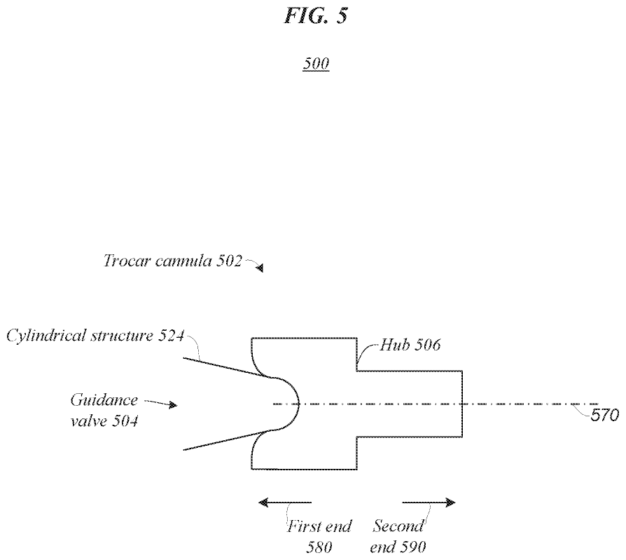

In various embodiments, guidance valve 104 may include a cylindrical or conical structure extending toward first end 180 (see e.g., FIG. 5). In various such embodiments the cylindrical or conical structure may be cut into two halves. For instance, a surgical instrument may be inserted in the cylindrical or conical structure of trocar cannula 102, and the cylindrical or conical structure may act as guidance as the surgical instrument passes through opening 105 and past hub 106. In one or more embodiments described herein, guidance valve 104 may guide a surgical instrument into alignment with entry axis 170 by redirecting forces used to insert the surgical instrument into trocar cannula 102 such that they force the surgical instrument toward the axis of the trocar cannula 102. In many embodiments, guidance valve 104 may be shaped such that little to no force used to insert the surgical instrument into trocar cannula 102 are redirected by the guidance valve 104 toward the first end 180. In other words, in several embodiments, little to no force may be reflected up the shaft of the surgical instrument when the surgical instrument comes into contact with guidance valve 104 during insertion through trocar cannula 102. In these and other ways trocar cannula 102 may prevent bending or kinking of surgical instruments utilizing trocar cannula 102 for access to a surgical site.

FIG. 2 illustrates an operating environment 200 that may be representative of various embodiments. Operating environment 200 may include an eye 220 in addition to trocar cannula 102. In one or more embodiments, the penetrating portion 110 of trocar cannula 102 may be utilized to gain and/or provide surgical tools with access to interior 214. In various embodiments, trocar cannula 102 may limit equalization between intraocular pressure 212 on the interior 214 of eye 220 and ambient pressure 216 on the exterior 218 of eye 220. Embodiments are not limited in this context.

As previously mentioned, in one or more embodiments, guidance valve 104 may prevent or limit equalization of different pressures to which the first and second ends 180, 190 are exposed (e.g., intraocular pressure 212 and ambient pressure 216). For example, guidance valve 104 may limit or prevent a drop in intraocular pressure 212. In one or more embodiments, guidance valve 104 may prevent or reduce leakage of a higher pressure at the surgical site, intraocular pressure 212, to a lower pressure external to the surgical site, ambient pressure 216. In various embodiments, trocar cannula 102 may include one or more slits or holes. In many embodiments, trocar cannula 102 may include a membrane with an opening that conforms to surgical instruments inserted therethrough to prevent or limit equalization of the intraocular pressure 212 and the ambient pressure 216. In some embodiments, guidance valve 104 may utilize an elastic material to conform with surgical instruments passed therethrough.

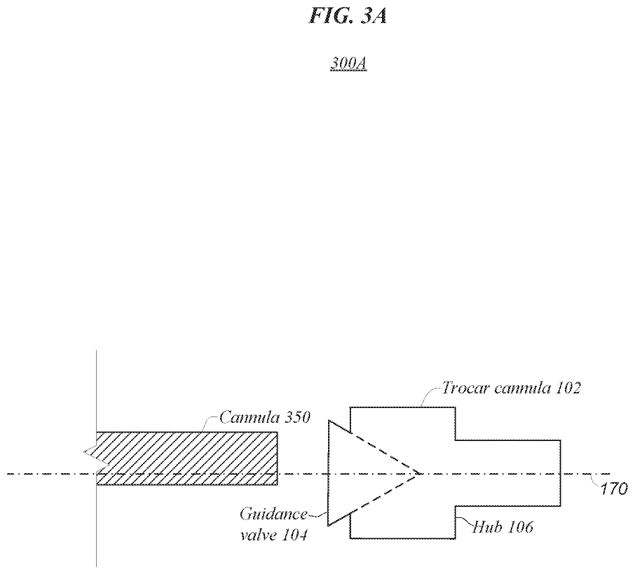

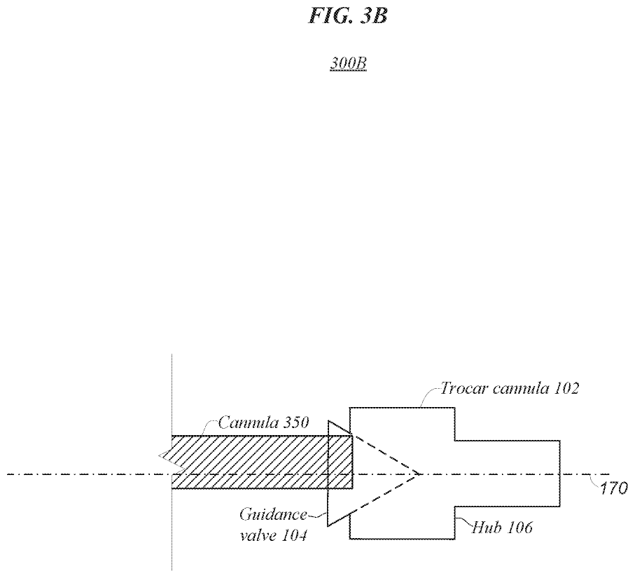

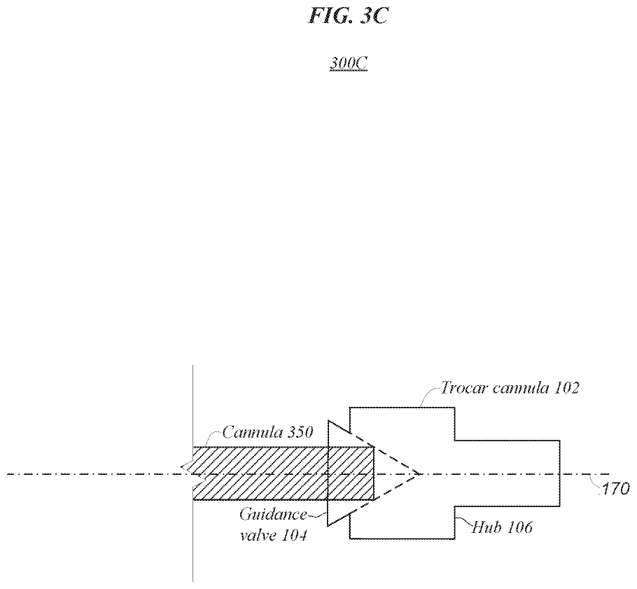

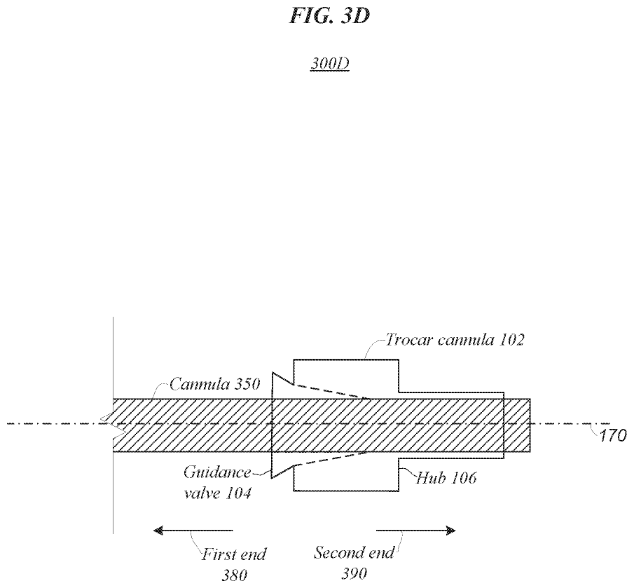

FIGS. 3A-3D illustrate an embodiment of a process that may be representative of various embodiments. In various embodiments, the process may include insertion of a surgical instrument tip 350 (e.g., a cannula) through trocar cannula 102. In the illustrated embodiments, the process may include a first, second, third, and fourth states 300A, 300B, 300C, 300D of trocar cannula 102 in conjunction with surgical instrument tip 350. In the first state 300A, surgical instrument tip 350 is misaligned with entry axis 170 of trocar cannula 102. In the second state 300B, surgical instrument tip 350 is inserted into trocar cannula 102 while still misaligned with entry axis 170. In one or more embodiments described herein, due to being misaligned with entry axis 170, surgical instrument tip 350 may contact a portion of guidance valve 104 that guides the surgical instrument tip 350 toward alignment with entry axis 170. In the third state 300C, insertion has continued such that guidance valve 104 has guided surgical instrument tip 350 into axial alignment with trocar cannula 102 along entry axis 170. In the fourth state 300D, guidance valve 104 has conformed to the shape of surgical instrument tip 350 to prevent or limit equalization of a pressure difference between the first and second ends 380, 390 of trocar cannula 102. Embodiments are not limited in this context.

In one or more embodiments, guidance valve 104 may align a surgical instrument with entry axis 170 such that a surgical instrument with a soft or flexible tip can be passed through trocar cannula 102 without becoming kinked, tangled and/or blocked. As previously mentioned, in various embodiments described herein, trocar cannula 102 may include guidance valve 104 that is shaped to promote alignment of a surgical instrument tip 350 (e.g., a cannula) with entry axis 170 of a trocar cannula 102. For instance, the guidance valve 104 may include a funnel shape to guide alignment of surgical instrument tip 350 with the entry axis 170 of the trocar cannula 102 to access a surgical site. In some instances, the guidance valve 104 may include a membrane with a concave surface shaped to guide axial alignment of surgical instrument tip 350 with trocar cannula 104. In some embodiments, surgical instrument tip 350 may include a soft or flexible tip. In some such embodiments, guidance valve 104 may be shaped to keep the soft or flexible portion of surgical instrument tip 350 straight as it is inserted through guidance valve 104.

FIG. 4 illustrates an operating environment 400 that may be representative of various embodiments. Operating environment 400 may include trocar cannula 402 with guidance valve 404 and hub 406. In various embodiments, trocar cannula 402, or one or more components thereof, may be the same or similar to another trocar cannula described herein, or one or more components thereof. For instance, trocar cannula 402 may include opening 105 or cylindrical or conical structure 524. In one or more embodiments described herein, guidance valve 404 may promote alignment of a surgical instrument with entry axis 470. In some embodiments, guidance valve 404 may include a hollow 422. In some embodiments, the hollow 422 may be cylindrical. In one or more embodiments, guidance valve 404 may include a concave surface exposed to ambient pressure. In some embodiments, guidance valve 404 may include a membrane. Embodiments are not limited in this context.

In one or more embodiments, insertion of a surgical instrument through trocar cannula 402 may be simplified and expedited with guidance valve 404. In some embodiments, guidance valve 404 may include a membrane with a cylindrical hollow (e.g., hollow 422) aligned with entry axis 470. In some embodiments, a diameter of the cylindrical hollow may decrease when moving towards hub 406. When a surgical instrument is inserted in the hollow proximate first end 480, the hollow provides guidance such that it can pass through trocar cannula 402 and out of second end 490 without becoming kinked or damaged. In one or more embodiments, a distance from guidance valve 404 to hub 406 may reach a minimum proximate entry axis 470. In these and other ways trocar cannula 402 may prevent bending or kinking of surgical instruments utilizing trocar cannula 402 for access to a surgical site.

FIG. 5 illustrates an operating environment 500 that may be representative of various embodiments. Operating environment 500 may include trocar cannula 502 with guidance valve 504 and hub 506. In various embodiments, trocar cannula 502, or one or more components thereof, may be the same or similar to another trocar cannula described herein, or one or more components thereof. For example, trocar cannula 502 may include hollow 422 or opening 105. In one or more embodiments described herein, guidance valve 504 may promote alignment of a surgical instrument with entry axis 570. In some embodiments, guidance valve 504 may include a cylindrical or conical structure 524 extending toward first end 580. In various embodiments, cylindrical or conical structure 524 may guide a surgical instrument into axial alignment with trocar cannula 502. Embodiments are not limited in this context.

In some embodiments, guidance valve 504 may include cylindrical or conical structure 524 extending toward first end 580. In some embodiments, the cylindrical or conical structure 524 may be cut into two portions (e.g., top and bottom halves). For instance, a surgical instrument may be inserted in the cylindrical or conical structure 524 of trocar cannula 502, and the cylindrical or conical structure 524 may act as guidance as the surgical instrument passes through trocar cannula 502 along entry axis 570. In these and other ways trocar cannula 502 may prevent bending or kinking of surgical instruments utilizing trocar cannula 502 for access to a surgical site.

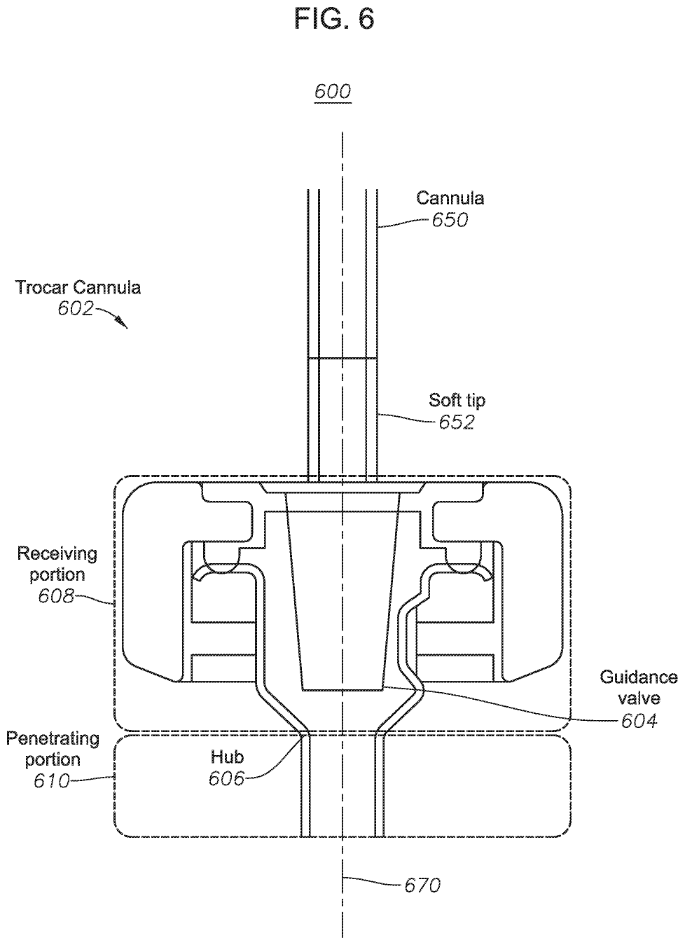

FIG. 6 illustrates an operating environment 600 that may be representative of various embodiments. Operating environment 600 may include trocar cannula 602 in conjunction with surgical instrument tip 650. In various embodiments, trocar cannula 602, or one or more components thereof, may be the same or similar to another trocar cannula described herein, or one or more components thereof. For example, trocar cannula 602 may include cylindrical or conical structure 524 or opening 105. In the illustrated embodiment, trocar cannula 602 includes receiving portion 608 with guidance valve 604 and penetrating portion 610 coupled via hub 606. In one or more embodiments described herein, guidance valve 604 may promote alignment of surgical instrument tip 650 with entry axis 670. In one or more embodiments, surgical instrument tip 650 may include soft tip 652. Embodiments are not limited in this context.

FIG. 7 illustrates an operating environment 700 that may be representative of various embodiments. Operating environment 700 may include trocar cannula 702. In various embodiments, trocar cannula 702, or one or more components thereof, may be the same or similar to another trocar cannula described herein, or one or more components thereof. For example, trocar cannula 702 may include hollow 422 or opening 105. In the illustrated embodiment, trocar cannula 702 includes receiving portion 708 with guidance valve 704 and penetrating portion 710 coupled via hub 706. In various embodiments described herein, guidance valve 704 may prevent or limit equalization of different pressures between the receiving portion 708 and penetrating portions 710 of trocar cannula 702. In one or more embodiments described herein, guidance valve 704 may promote alignment of a surgical instrument with entry axis 770. Embodiments are not limited in this context.

In many embodiments, the guidance valve 704 may include a partially spherical shape. In many such embodiments, the partially spherical shape may utilize intraocular pressure to limit an amount of intraocular pressure lost. For example, backpressure (see e.g., white arrows of FIG. 7) on the guidance valve 104 created by the intraocular pressure may support self-sealing of the guidance valve with a surgical instrument, such as a backflush or soft tip cannula. In various embodiments, guidance valve 704 may comprise one or more of an elastic material, silicon, polyurethane, and polyimide. Other materials area also contemplated. In one or more embodiments, guidance valve 704 may prevent or reduce leakage of a higher pressure at the surgical site to a lower pressure external to the surgical site, such as loss of intraocular pressure within an eye. In various embodiments, guidance valve 104 may include one or more slits or holes. In many embodiments, guidance valve 104 may include a membrane that conforms to surgical instruments inserted therethrough to prevent or limit equalization of different pressures to which the receiving and penetrating portions 708, 710 are exposed.

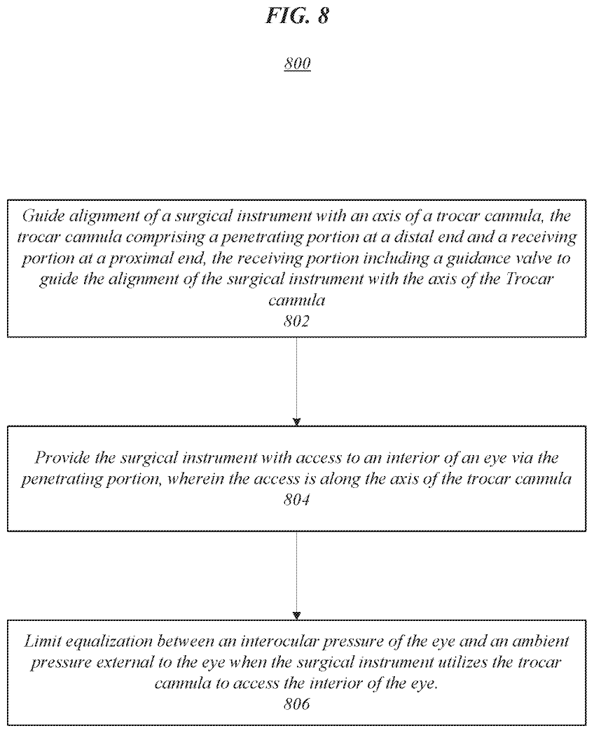

FIG. 8 illustrates an embodiment of a flow or method 800, which may be representative of operations that may be executed in various embodiments in conjunction with accessing a surgical site via a trocar cannula with a guidance valve (e.g., trocar cannula 104). The flow or method 800 may be representative of some or all of the operations that may be performed to utilize one or more trocar cannulas described herein. The embodiments are not limited in this context.

The flow 800 may begin at block 802. At block 802 a surgical instrument may be guided into alignment with the axis of a trocar cannula. In various embodiments, the trocar cannula may include a penetrating portion at a distal end and a receiving portion at a proximal end, and the receiving portion may include a guidance valve that guides the alignment of the surgical instrument with the axis of the trocar cannula. For instance, trocar cannula 102 may include receiving portion 108 at first end 180 and penetrating portion 110 at second end 190. In various examples, guidance valve 404 of trocar cannula 402 may guide at least a portion of surgical instrument tip 350, such as a soft tip of the surgical instrument tip 350, into alignment with entry axis 470. In various such examples, hollow 422 of trocar cannula 402 may include one or more of a conical, tapered, convex, or concave shape to position surgical instrument tip 350 in alignment with entry axis 470 prior to passing hub 406. In various embodiments, guidance valve 104 may position at least a portion of surgical instrument tip 350, such as a soft tip, in alignment with entry axis 470 without kinking or causing damage to surgical instrument tip 350. In some embodiments, the guidance valve 104 may keep a soft tip of surgical instrument tip 350 straight as the surgical instrument tip 350 is inserted therethrough.

Proceeding to block 804, the penetrating portion of the surgical instrument may provide the surgical instrument with access to an interior of an eye along the axis of the trocar cannula. For instance, penetrating portion 110 of trocar cannula 102 may provide surgical instrument tip 350 with access to the interior 214 of eye 220. Continuing to block 806, equalization between an intraocular pressure of the eye and an ambient pressure external to the eye may be limited when the surgical instrument utilizes the trocar cannula to access the interior of the eye. For example, trocar cannula 102 may limit equalization between intraocular pressure 212 on the interior 214 of eye 22 and ambient pressure 216 on the exterior 218 of eye 220. In such examples, guidance valve 104 may seal around a surgical instrument tip 650, such as a soft tip cannula, when the surgical instrument tip is inserted through trocar cannula 102 to limit the exchange of pressure between the interior 214 and exterior 218 of eye 220.

The following examples pertain to further embodiments, from which numerous permutations and configurations will be apparent.

Example 1 is an apparatus for use in ocular surgery, comprising: a trocar cannula with a penetrating portion at a distal end and a receiving portion at a proximal end, the penetrating portion configured to extend into an interior of an eye, the trocar cannula configured for providing a surgical instrument with access to the interior of the eye through the receiving portion and the penetrating portion, wherein the access is along an entry axis of the trocar cannula; and a guidance valve in the receiving portion shaped to guide alignment of at least one portion of the surgical instrument with the entry axis of the trocar cannula, wherein the guidance valve limits equalization between an intraocular pressure of the eye and an ambient pressure external to the eye when the surgical instrument utilizes the trocar cannula to access the interior of the eye.

Example 2 includes the subject matter of Example 1, wherein the guidance valve comprises a cylindrical hollow that decreases in diameter toward the distal end to guide alignment of the surgical instrument with the entry axis of the trocar cannula.

Example 3 includes the subject matter of Example 1, wherein the guidance valve comprises a concave surface exposed to the ambient pressure, the concave surface shaped to guide axial alignment of the surgical instrument with the trocar cannula.

Example 4 includes the subject matter of Example 1, wherein the at least one portion of the surgical instrument comprise a soft tip of a cannula and the guidance valve is shaped to keep the soft tip straight when the surgical instrument utilizes the trocar cannula to access the interior of the eye.

Example 5 includes the subject matter of Example 1, wherein the guidance valve comprises a convex surface exposed to the intraocular pressure and the intraocular pressure pushes the convex surface against the surgical instrument to limit equalization between the intraocular pressure and the ambient pressure when the surgical instrument utilizes the trocar cannula to access the interior of the eye.

Example 6 includes the subject matter of Example 1, wherein the guidance valve comprises a convex surface exposed to the intraocular pressure and the intraocular pressure causes the convex surface to limit equalization between the intraocular pressure of the eye and the ambient pressure external to the eye when the surgical instrument is removed from the trocar cannula.

Example 7 includes the subject matter of Example 1, wherein the surgical instrument comprises a flexible tip.

Example 8 includes the subject matter of Example 7, wherein the surgical instrument tip comprises a cannula.

Example 9 includes the subject matter of Example 1, wherein the guidance valve comprises silicon, polyurethane, or polyimide shaped to guide axial alignment of the surgical instrument with the trocar cannula.

Example 10 includes the subject matter of Example 1, wherein a distance from the guidance valve to the axis of the trocar cannula decreases toward the distal end to guide axial alignment of the surgical instrument with the trocar cannula.

Example 11 includes the subject matter of Example 1, the guidance valve comprising an opening that conforms to surgical instruments passed therethrough to limit equalization between the intraocular pressure and the ambient pressure when the surgical instrument utilizes the trocar cannula to access the interior of the eye.

Example 12 includes the subject matter of Example 11, wherein the opening comprises one or more slits in a membrane.

Example 13 includes the subject matter of Example 1, wherein the guidance valve comprises an opening that conforms to create a seal between the guidance valve and the surgical instrument to limit equalization between the intraocular pressure and the ambient pressure when the surgical instrument utilizes the trocar cannula to access the interior of the eye.

Example 14 includes the subject matter of Example 1, wherein the intraocular pressure pushes the guidance valve against the surgical instrument to limit equalization between the intraocular pressure and the ambient pressure when the surgical instrument utilizes the trocar cannula to access the interior of the eye.

Example 15 includes the subject matter of Example 1, wherein the guidance valve comprises a funnel shaped to guide axial alignment of the surgical instrument with the trocar cannula.

Example 16 includes the subject matter of Example 1, wherein the trocar cannula comprises a valved trocar cannula.

Example 17 includes the subject matter of Example 1, wherein the at least one portion of the surgical instrument comprise a soft tip of a cannula and the guidance valve is shaped to keep the soft tip straight when the surgical instrument utilizes the trocar cannula to access the interior of the eye.

Example 18 includes the subject matter of Example 1, wherein the guidance valve comprises a convex surface exposed to the intraocular pressure and the intraocular pressure causes the convex surface to limit equalization between the intraocular pressure of the eye and the ambient pressure external to the eye when the surgical instrument is removed from the trocar cannula.

Example 19 is a method, comprising: guiding alignment of at least one portion of a surgical instrument with an entry axis of a trocar cannula, the trocar cannula comprising a penetrating portion at a distal end and a receiving portion at a proximal end, the receiving portion including a guidance valve to guide the alignment of the surgical instrument with the entry axis of the trocar cannula; providing the surgical instrument with access to an interior of an eye via the penetrating portion, wherein the access is along the entry axis of the trocar cannula; and limiting equalization between an intraocular pressure of the eye and an ambient pressure external to the eye when the surgical instrument utilizes the trocar cannula to access the interior of the eye.

Example 20 includes the subject matter of Example 19, wherein the guidance valve comprises a cylindrical hollow that decreases in diameter toward the distal end to guide alignment of the surgical instrument with the entry axis of the trocar cannula.

Example 21 includes the subject matter of Example 19, wherein the guidance valve comprises a concave surface exposed to the ambient pressure, the concave surface shaped to guide axial alignment of the surgical instrument with the trocar cannula.

Example 22 includes the subject matter of Example 19, wherein the guidance valve comprises a cylindrical port in axial alignment with the trocar cannula to guide axial alignment of the surgical instrument with the trocar cannula.

Example 23 includes the subject matter of Example 19, wherein the guidance valve comprises a convex surface exposed to the intraocular pressure and the intraocular pressure pushes the convex surface against the surgical instrument to limit equalization between the intraocular pressure and the ambient pressure when the surgical instrument utilizes the trocar cannula to access the interior of the eye.

Example 24 includes the subject matter of Example 19, wherein the surgical instrument comprises a flexible tip.

Example 25 includes the subject matter of Example 24, wherein the surgical instrument tip comprises a cannula.

Example 26 includes the subject matter of Example 19, wherein the guidance valve comprises silicon, polyurethane, or polyimide shaped to guide axial alignment of the surgical instrument with the trocar cannula.

Example 27 includes the subject matter of Example 19, wherein a distance from the guidance valve to the axis of the trocar cannula decreases toward the distal end to guide axial alignment of the surgical instrument with the trocar cannula.

Example 28 includes the subject matter of Example 19, the guidance valve comprising an opening that conforms to surgical instruments passed therethrough to limit equalization between the intraocular pressure and the ambient pressure when the surgical instrument utilizes the trocar cannula to access the interior of the eye.

Example 29 includes the subject matter of Example 28, wherein the opening comprises one or more slits in a membrane.

Example 30 includes the subject matter of Example 19, wherein the guidance valve comprises an opening that conforms to create a seal between the guidance valve and the surgical instrument to limit equalization between the intraocular pressure and the ambient pressure when the surgical instrument utilizes the trocar cannula to access the interior of the eye.

Example 31 includes the subject matter of Example 19, wherein the intraocular pressure pushes the guidance valve against the surgical instrument to limit equalization between the intraocular pressure and the ambient pressure when the surgical instrument utilizes the trocar cannula to access the interior of the eye.

Example 32 includes the subject matter of Example 19, wherein the guidance valve comprises a funnel shaped to guide axial alignment of the surgical instrument with the trocar cannula.

Example 33 includes the subject matter of Example 19, wherein the trocar cannula comprises a valved trocar cannula.

Example 34 includes the subject matter of Example 19, wherein the at least one portion of the surgical instrument comprises a soft tip of a cannula and the guidance valve is shaped to keep the soft tip straight when the surgical instrument utilizes the trocar cannula to access the interior of the eye.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner, and may generally include any set of one or more limitations as variously disclosed or otherwise demonstrated herein.

1. An apparatus for use in ocular surgery, comprising:
a trocar cannula with a penetrating portion at a distal end and a receiving portion at a proximal end, the penetrating portion configured to extend into an interior of an eye, the trocar cannula configured for providing a surgical instrument with access to the interior of the eye through the receiving portion and the penetrating portion, wherein the access is along an entry axis of the trocar cannula; and
a guidance valve in the receiving portion shaped to guide alignment of at least one portion of the surgical instrument with the entry axis of the trocar cannula, wherein the guidance valve limits equalization between an intraocular pressure of the eye and an ambient pressure external to the eye when the surgical instrument utilizes the trocar cannula to access the interior of the eye.

2. The apparatus of claim 1, wherein the guidance valve comprises a cylindrical hollow that decreases in diameter toward the distal end to guide alignment of the surgical instrument with the entry axis of the trocar cannula.

3. The apparatus of claim 1, wherein the guidance valve comprises a concave surface exposed to the ambient pressure, the concave surface shaped to guide axial alignment of the surgical instrument with the trocar cannula.

4. The apparatus of claim 1, wherein the at least one portion of the surgical instrument comprise a soft tip of a cannula and the guidance valve is shaped to keep the soft tip straight when the surgical instrument utilizes the trocar cannula to access the interior of the eye.

5. The apparatus of claim 1, wherein the guidance valve comprises a convex surface exposed to the intraocular pressure and the intraocular pressure pushes the convex surface against the surgical instrument to limit equalization between the intraocular pressure and the ambient pressure when the surgical instrument utilizes the trocar cannula to access the interior of the eye.

6. The apparatus of claim 1, wherein the guidance valve comprises a convex surface exposed to the intraocular pressure and the intraocular pressure causes the convex surface to limit equalization between the intraocular pressure of the eye and the ambient pressure external to the eye when the surgical instrument is removed from the trocar cannula.

7. The apparatus of claim 1, wherein the surgical instrument comprises a flexible tip.

8. The apparatus of claim 7, wherein the surgical instrument tip comprises a cannula.

9. The apparatus of claim 1, wherein the guidance valve comprises silicon, polyurethane, or polyimide shaped to guide axial alignment of the surgical instrument with the trocar cannula.

10. The apparatus of claim 1, wherein a distance from the guidance valve to the axis of the trocar cannula decreases toward the distal end to guide axial alignment of the surgical instrument with the trocar cannula.

11. The apparatus of claim 1, the guidance valve comprising an opening that conforms to surgical instruments passed therethrough to limit equalization between the intraocular pressure and the ambient pressure when the surgical instrument utilizes the trocar cannula to access the interior of the eye.

12. The apparatus of claim 11, wherein the opening comprises one or more slits in a membrane.

13. The apparatus of claim 1, wherein the guidance valve comprises an opening that conforms to create a seal between the guidance valve and the surgical instrument to limit equalization between the intraocular pressure and the ambient pressure when the surgical instrument utilizes the trocar cannula to access the interior of the eye.

14. The apparatus of claim 1, wherein the intraocular pressure pushes the guidance valve against the surgical instrument to limit equalization between the intraocular pressure and the ambient pressure when the surgical instrument utilizes the trocar cannula to access the interior of the eye.

15. The apparatus of claim 1, wherein the guidance valve comprises a funnel shaped to guide axial alignment of the surgical instrument with the trocar cannula.

16. The apparatus of claim 1, wherein the trocar cannula comprises a valved trocar cannula.

17. A method, comprising:
guiding alignment of at least one portion of a surgical instrument with an entry axis of a trocar cannula, the trocar cannula comprising a penetrating portion at a distal end and a receiving portion at a proximal end, the receiving portion including a guidance valve to guide the alignment of the surgical instrument with the entry axis of the trocar cannula;
providing the surgical instrument with access to an interior of an eye via the penetrating portion, wherein the access is along the entry axis of the trocar cannula; and
limiting equalization between an intraocular pressure of the eye and an ambient pressure external to the eye when the surgical instrument utilizes the trocar cannula to access the interior of the eye.

18. The method of claim 16, wherein the guidance valve comprises a cylindrical hollow that decreases in diameter toward the distal end to guide alignment of the surgical instrument with the entry axis of the trocar cannula.

19. The method of claim 16, wherein the guidance valve comprises a concave surface exposed to the ambient pressure, the concave surface shaped to guide axial alignment of the surgical instrument with the trocar cannula.

20. The method of claim 16, wherein the at least one portion of the surgical instrument comprises a soft tip of a cannula and the guidance valve is shaped to keep the soft tip straight when the surgical instrument utilizes the trocar cannula to access the interior of the eye.
